(19) United States
(12) Patent Application Publication
(10) Pub. No.: US 2013/0316418 A1
(43) Pub. Date: Nov. 28, 2013

(54) METHOD FOR PRODUCING 2,3-BUTANEDIOL BY FERMENTATION

(71) Applicant: Rupert Pfaller, Muenchen (DE)

(72) Inventor: Rupert Pfaller, Muenchen (DE)

(73) Assignee: WACKER CHEMIE AG, Muenchen (DE)

(21) Appl. No.: 13/979,025

(22) PCT Filed: Jan. 30, 2012
(86) PCT No.: PCT/EP12/51428

§ 371 Date: Jul. 10, 2013

(30) Foreign Application Priority Data
Jan. 31, 2011 (DE) 10 2011 003 387.4

(51) Int. Cl. C12P 7/18

(52) U.S. Cl. CPC C12P 7/18
USPC 435/158; 435/252.3

(57) ABSTRACT

The invention relates to a production strain for producing 2,3-butanediol. Said production strain can be produced from an original strain selected from the genes Klebsiella, Raoultella, Paenibacillus and Bacillus and has an acetoin reductase activity at least 3.6-34.8 times higher than the original strain. The invention also relates to the production of 2,3-butanediol by means of said production strain.

The invention relates to a method for producing 2,3-butanediol (2,3-BDL) by fermentation by means of an improved microorganism strain, which has an acetoin reductase (synonymous with 2,3-butanediol dehydrogenase) activity increased 3.6 to 34.8 times compared to the non-improved original strain.

Because of increasing crude oil prices, the production costs of the base materials for the chemical industry obtained from them petrochemically are also increasing. This is resulting in a growing interest in alternative production methods for chemical base materials, especially on the basis of renewable raw materials.

Examples of chemical base materials (so-called chemical synthesis building blocks) from renewable raw materials are ethanol (C2 building block), glycerine, 1,3-propanediol, 1,2-propanediol (C3 building blocks) or succinic acid, 1-butanol, 2-butanol, 1,4-butanediol or also 2,3-butanediol (C4 building blocks). These chemical building blocks are the biogenic starting compounds from which further base chemicals can then be produced by chemical routes. The prerequisite for this is the inexpensive production of the particular synthesis building blocks from renewable raw materials by fermentation. Decisive cost factors are on the one hand the availability of suitable cheap renewable raw materials and on the other hand efficient microbial fermentation methods which efficiently convert these raw materials into the desired chemical base material. Here it is decisive that the microorganisms used produce the desired product in high concentration and with little by-product formation from the biogenic raw material. The optimization of the microorganisms designated as production strains with regard to productivity and profitability is for example achieved by metabolic engineering.

A C4 building block accessible by a fermentative route is 2,3-butanediol. The state of the art on the production of 2,3-butanediol by fermentation is summarized in Celinska and Grajek (Biotechnol. Advances (2009) 27: 715-725). 2,3-butanediol is a possible starting product for products of petrochemistry with four C atoms (C4 building blocks) such as acetoin, diacetyl, 1,3-butadiene and 2-butanone (methyl ethyl ketone, MEK). In addition, products with two C atoms (C2 building blocks) such as acetic acid (DE 102010001399) and, derived therefrom, acetaldehyde, ethanol and even ethylene are accessible. Through dimerization of 2,3-butanediol, C8 compounds, which are used for example as fuel in the air travel sector, are also feasible.

The biosynthetic pathway to 2,3-butanediol trodden by various microorganisms is known (see review by Celinska and Grajek, Biotechnol. Advances (2009) 27: 715-725) and leads from the central metabolic product pyruvate via the three following enzymatic steps to 2,3-butanediol:

Reaction of acetolactate synthase: formation of acetolactate from two molecules of pyruvate with elimination of CO₂.

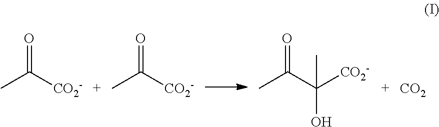

Reaction of acetolactate decarboxylase: decarboxylation of acetolactate to acetoin.

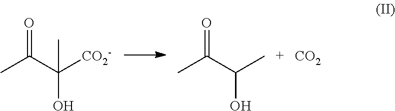

Reaction of acetoin reductase (2,3-butanediol dehydrogenase): NADH-dependent reduction of acetoin to 2,3-butanediol.

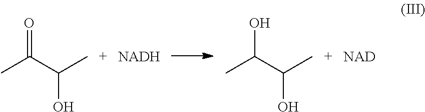

Various natural producers of 2,3-butanediol are known, e.g. from the genera Klebsiella, Raoultella, Enterobacter, Aerobacter, Aeromonas, Serratia, Bacillus, Paenibacillus, Lactobacillus, Lactococcus etc. However, yeasts are also known as producers (e.g. baker's yeast). Most bacterial producers are microorganisms of biosafety level S2, and can thus not be used on an industrial scale without laborious and costly industrial safety measures (concerning this and other microbial 2,3-butanediol producers see review by Celinska and Grajek, Biotechnol. Advances (2009) 27: 715-725). This would also apply for not previously described genetically optimized production strains based on these strains.

Hence only production strains of the biosafety level S1 are a possibility for cost-efficient industrial scale 2,3-BDL production. However, sufficiently high 2,3-BDL yields have not previously been described for these. Known 2,3-BDL production strains from the biosafety level S1 are strains of the species Klebsiella terrigena, Klebsiella planticola and strains of the genus Bacillus (or Paenibacillus) such as Bacillus polymyxa or Bacillus licheniformis. In the technical literature (Drancourt et al., Int. J. Syst. Evol. Microbiol. (2001), 51: 925-932) as a result of a change in taxonomic nomenclature, the species Klebsiella terrigena and Klebsiella planticola are also synonymously designated as Raoultella terrigena and Raoultella planticola. These are strains of the same species.

For these strains classified in safety level S1, 2,3-butanediol yields of not more than 57 g/l (637 mM, production time 60 hrs) have previously been reported (Nakashimada et al., J. Bioscience and Bioengineering (2000) 90: 661-664). These yields are far too low for profitable production. >80 g/l 2,3-butanediol (fermentation time maximum 72 hrs), preferably >100 g/l 2,3-butanediol, is regarded as the minimum fermentation yield for profitable production.

A current definition of the term “biosafety level” and the classification into various safety levels can for example be found in “Biosafety in Microbiological and Biomedical Laboratories”, p. 9ff. (Table 1), Centers for Disease Control and Prevention (U.S.) (Editor), Public Health Service (U.S.) (Editor), National Institutes of Health (Editor), Publisher: U.S. Dept. of Health and Human Services; 5, 5th edition, Revised December 2009 edition (Mar. 15, 2010); ISBN-10: 0160850428.

The increasing of the acetoin reductase activity (or 2,3-butanediol dehydrogenase activity) in bacteria is described in US 2010/0112655, WO 2009/151342 and WO 99/54453.

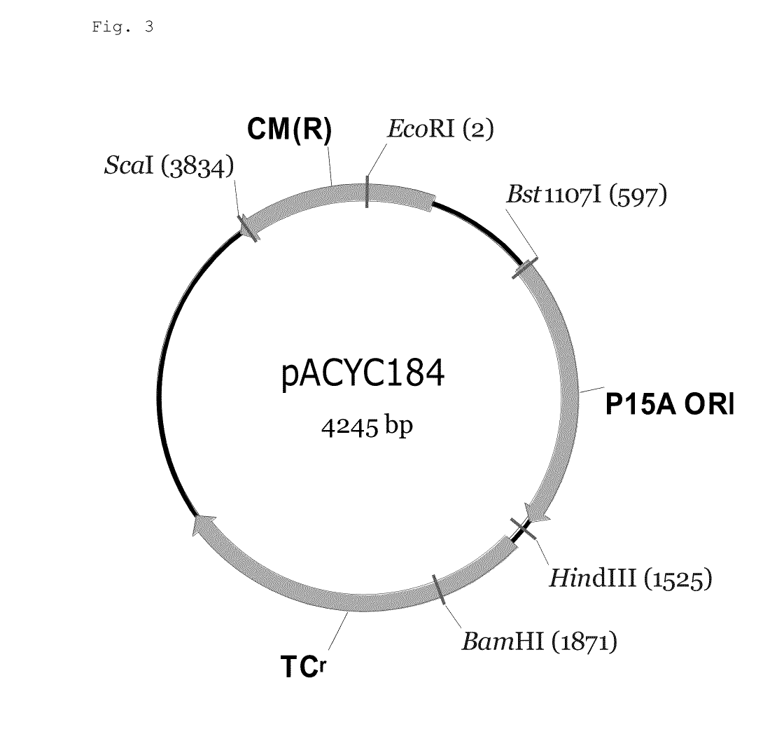

The heterologous expression of a gene in lactic acid bacteria e.g. of the species Lactobacillus plantarum which codes for a protein with butanediol dehydrogenase activity is described in US 2010/0112655. However, the prerequisite here for successful 2,3-butanediol production is that the production strain is free from activity of the enzyme lactate dehydrogenase (LDH). FIG. 3 in US 2010/0112655 shows that a recombinant L. plantarum strain expressing acetoin reductase from Klebsiella pneumoniae (Bud C) and alcohol dehydrogenase (Sad B) from Achromobacter xylosoxidans produces no 2,3-butanediol, just as little as an L. plantarum mutant with an LDH deletion. Only the combination of a recombinant L. plantarum mutant with LDH deletion and at the same time heterologous expression of Bud C and Sad B leads to the production of small quantities of 2,3-butanediol with a yield of 80 mM (corresponds to 7.2 g/l). Based on the total yield of products formed determined in molarity (see FIG. 3 in US 2010/0112655) the proportion of 2,3-butanediol was 49 mol. % of the products measured which were formed from glucose. The selectivity of the conversion of glucose into 2,3-butanediol is thus comparatively low.

For those skilled in the art it follows from this that the simple overexpression of an acetoin reductase does not lead to an increase in the 2,3-butanediol yield in a production strain, but rather that for this a laborious strain optimization is necessary beforehand, during which on the one hand one or more interfering LDH activities (in the case of L. plantarum Ldh D and Ldh L1) must be removed and in addition an alcohol dehydrogenase activity must be introduced recombinantly. Furthermore, a yield of 49 mol. % 2,3-butanediol is too low for profitable exploitation since more than half of the glucose used results in the formation of by-products.

WO 2009/151342 discloses the production of 2,3-BDL by anaerobic fermentation with the bacterium Clostridium autoethanogenum and carbon monoxide as the C source. The maximum 2,3-BDL yields achieved were 9.27 g/l after a fermentation time of 14 days. In the non-genetically modified strains, as a measure of the expression intensity, the RNA content of the 2,3-butanediol dehydrogenase in 2,3-BDL-producing preparations (caused by carbon monoxide gassing in excess) in comparison to an acetate-producing preparation (limited carbon monoxide gassing) was determined and an induction of the RNA of the wild type gene of 2,3-butanediol dehydrogenase by the factor 7.64 (corresponding to 2,3-BDL production of 8.67 g/l after 13 days) was measured. The induction of the gene expression was achieved by increased gassing with carbon monoxide. It was not disclosed whether an increase in the acetoin reductase enzyme activity was also connected with the gene expression and how great the supposed increase in the enzyme activity was in comparison to the starting condition. It is known to those skilled in the art that no linear relationship exists between transcription (RNA content) and translation (enzyme content), so that a 7.64-fold increase in the acetoin reductase RNA in WO 2009/151342 does not necessarily correspond to a 7.64-fold increase in the acetoin reductase enzyme activity. With the chosen preparation, it cannot be distinguished whether the increased 2,3-BDL production occurred because of the increased input of the C source carbon monoxide or because of intensified expression of the 2,3-butanediol dehydrogenase enzyme. Carbon monoxide is toxic under conditions of aerobic metabolism, so that no general teaching as to production under aerobic or microaerobic conditions for increasing the 2,3-butanediol production as a result of the combination of gassing with carbon monoxide and increased 2,3-butanediol dehydrogenase activity can be deduced from the special situation of the anaerobic fermentation of C. autoethanogenum. Furthermore, 2,3-BDL yields of 8.67 g/l in 13 days are far from sufficient for profitable production, especially taking account of the increased technical cost of an anaerobic fermentation on the industrial scale.

WO 99/54453 discloses lactic acid bacteria with 10-fold increased activity either of diacetyl reductase, acetoin reductase, or 2,3-butanediol dehydrogenase. In this case, surprisingly, only an effect on the diacetyl content of the lactic acid bacteria used as starter cultures in the foodstuffs industry was observed. It was however not disclosed whether increased activity of the acetoin reductase, or 2,3-butanediol dehydrogenase, had an effect on the 2,3-BDL content of the lactic acid bacterial cultures. Nor was it disclosed whether the strains disclosed in the invention still produce 2,3-BDL at all, or whether the 10-fold overexpression of the acetoin reductase resulted in an increase in the 2,3-BDL production. As already discussed for US 2010/0112655, in the presence of the lactic acid-producing enzyme lactate dehydrogenase, lactic acid bacteria produce no 2,3-BDL even after overexpression of the acetoin reductase, so that those skilled in the art also expect no significant production of 2,3-BDL for the lactic acid strains disclosed in WO 99/54453.

The invention disclosed in WO 99/54453 is limited to lactic acid bacteria. These are novel starter cultures for the foodstuffs industry and the foodstuffs produced therefrom are not intended to contain an increased content of 2,3-butanediol. It is thus not obvious to those skilled in the art that an increase in the acetoin reductase activity necessarily results in an increased 2,3-BDL yield.

The purpose of the invention was to provide production strains for producing 2,3-butanediol which enable markedly higher 2,3-butanediol yields than the original strain.

The problem was solved by means of a production strain which is producible from an original strain, characterized in that the original strain is selected from the genera Klebsiella, Raoultella, Paenibacillus and Bacillus and the production strain has an acetoin reductase activity lying at least 3.6 to 34.8 times higher than the original strain.

In the present invention, a distinction is made between an original strain and a production strain. The original strain can be a wild type strain which is not further optimized, but is capable of 2,3-butanediol production, or a wild type strain which is already further optimized. However, in a wild type strain which is already further optimized (e.g. effected through a genetic engineering operation) the acetoin reductase activity is not affected by the optimization.

In the sense of the present invention, a production strain should be understood to mean an original strain optimized as regards 2,3-butanediol production, which is characterized by increased activity of the enzyme acetoin reductase (AR) in comparison to the original strain. The production strain is preferably produced from the original strain. If an already optimized original strain is to be improved again by an increase in the acetoin reductase activity, then it is naturally also possible first to increase the acetoin reductase activity in an unimproved strain and then to introduce further improvements.

Here the increase in the acetoin reductase activity in the production strain can be caused by any mutation in the genome of the original strain (e.g. a mutation increasing the promoter activity), a mutation in the acetoin reductase gene increasing the enzyme activity or by overexpression of a homologous or else also heterologous acetoin reductase gene in the original strain.

Preferably the overexpression is of a homologous or of a heterologous acetoin reductase gene in the original strain.

Preferably, the acetoin reductase activity is increased by at least the factor 3.6 to 20 and particularly preferably by the factor 3.6 to 10.

Particularly preferably, this increased acetoin reductase activity is achieved by an increased expression of a homologous or heterologous gene coding for an acetoin reductase enzyme compared to the original strain.

The original strain can be any 2,3-butanediol-producing strain of the safety level S1.

Preferably it is a strain of the species Klebsiella (Raoultella) terrigena, Klebsiella (Raoultella) planticola, Bacillus (Paenibacillus) polymyxa or Bacillus licheniformis wherein a strain of the species Klebsiella (Raoultella) terrigena or Klebsiella (Raoultella) planticola is again preferable.

Particularly preferably, it is an original strain and a production strain classified in the safety level S1 and among these, more preferably, strains of the species Klebsiella (Raoultella) terrigena or Klebsiella (Raoultella) planticola.

According to the state of the art (see US 2010/0112655, WO 2009/151342 and WO 99/54453) those skilled in the art had to assume that the overexpression of the acetoin reductase cannot cause any improvement in the 2,3-butanediol production in a production strain. In the context of experiments which led to the present invention, it was surprisingly found that through a limited (3.6 to 34.8-fold) increased acetoin reductase activity in a production strain the yield of 2,3-butanediol can be significantly increased. Preferably, the increased acetoin reductase activity is achieved by recombinant overexpression of an acetoin reductase in a production strain.

As shown in the examples of the present application, an overexpression of the acetoin reductase by the factor 3.6 to up to a factor of 34.8 (see Example 3) is suitable for increasing the 2,3-butanediol yield (determined as 2,3-butanediol volume yield in g/l) in the fermentation by more than 20%, preferably more than 30%, particularly preferably more than 40% and especially preferably by more than 100%.

As further disclosed in Example 4, the recombinant overexpression of the acetoin reductase also enables an increase in the 2,3-butanediol yield in the shake flask by 20-29%.

The acetoin reductase (AR) can be any gene-coded enzyme which causes the synthesis of 2,3-butanediol from acetoin according to formula (III) by oxidation of the cofactor NADH (or also NADPH) to NAD (or NADP).

In a preferable embodiment, the gene of the acetoin reductase derives from a bacterium of the genus Klebsiella (Raoultella) or Bacillus (Paenibacillus).

In a particularly preferable embodiment, the gene of the acetoin reductase derives from a strain of the species Klebsiella terrigena, Klebsiella planticola, Bacillus polymyxa or Bacillus licheniformis and in particular from a strain of the species Klebsiella terrigena, Klebsiella planticola or Bacillus licheniformis. These strains, including also the strain Klebsiella terrigena DSM 2687 used in the present invention, are all commercially available e.g. at the DSMZ Deutsche Sammlung von Mikroorganismen and Zellkulturen GmbH (Braunschweig).

The strain according to the invention thus makes it possible to increase the production of 2,3-butanediol by fermentation. The invention thus enables not only the production of 2,3-butanediol but also the production of other metabolic products which can be derived from 2,3-butanediol. These metabolic products include diacetyl, acetoin, ethanol and acetic acid.

In a preferable embodiment, a production strain according to the invention is also characterized in that it was produced from an original strain, as defined in the application, and produces an acetoin reductase in recombinant form with the result that its 2,3-BDL production (volume production expressed in g/l 2,3-BDL) is increased compared to the non-genetically optimized original strain by at least 20%, preferably 30%, particularly preferably 40% and especially preferably by 100%, wherein the 2,3-butanediol yield of the original strain is at least 80 g/l.

The production strain according to the invention is preferably produced by introduction of a gene construct into one of said original strains.

The gene construct in its simplest form is defined as consisting of the acetoin reductase structural gene, operatively linked to which a promoter is positioned upstream. Optionally, the gene construct can also comprise a terminator which is positioned downstream from the acetoin reductase structural gene. A strong promoter which leads to strong transcription is preferred. Preferable among the strong promoters is the so-called “Tac promoter” familiar to those skilled in the art from the molecular biology of E. coli.

The gene construct can in a manner known per se be present in the form of an autonomously replicating plasmid, wherein the copy number of the plasmid can vary. A large number of plasmids, which depending on their genetic structure can replicate in autonomous form in a given production strain are known to those skilled in the art.

The gene construct can however also be integrated in the genome of the production strain, wherein any gene location along the genome is suitable as an integration site.

The gene construct, either in plasmid form or with the purpose of genomic integration, is introduced into the production strain in a manner known per se by genetic transformation. Various methods of genetic transformation are known to those skilled in the art (Aune and Aachmann, Appl. Microbiol. Biotechnol. (2010) 85: 1301-1313), including for example electroporation.

For the selection of transformed production strains, the gene construct, also in known manner, contains a so-called selection marker for the selection of transformants with the desired gene construct. Known selection markers are selected from so-called antibiotic resistance markers or from the selection markers complementing an auxotrophy.

Antibiotic resistance markers, particularly preferably those which impart resistance towards antibiotics selected from ampicillin, tetracycline, kanamycin, chloramphenicol or zeocin, are preferable.

In a preferable embodiment, a production strain according to the invention thus contains the gene construct, either in plasmid form or integrated into the genome, and produces an acetoin reductase enzyme in recombinant form. The recombinant acetoin reductase enzyme is capable of producing 2,3-butanediol from acetoin, with oxidation of NADH (or alternatively also NADPH) to NAD (or alternatively to NADP). Now it has surprisingly been found that by suitable recombinant overexpression of the acetoin reductase enzyme in the production strain the 2,3-butanediol yield can be significantly increased compared to the original strain.

The original strain can be a not further optimized wild type strain. The original strain can however already have been previously optimized, and be further optimized as an acetoin reductase-producing production strain according to the invention. The optimization of an acetoin reductase-producing production strain according to the invention comprised by the invention can on the one hand be effected by mutagenesis and selection of mutants with improved production properties. The optimization can however also be effected genetically by additional expression of one or more genes which are suitable for improving the production properties. Examples of such genes are the already mentioned 2,3-butanediol biosynthesis genes acetolactate synthase and acetolactate decarboxylase. These genes can be expressed in the production strain in a manner known per se each as individual gene constructs or else also combined as one expression unit (as a so-called operon). Thus for example it is known that in Klebsiella terrigena all three biosynthesis genes for 2,3-butanediol (so-called BUD operon, Blomqvist et al., J. Bacteriol. (1993) 175: 1392-1404), and in strains of the genus Bacillus the genes of acetolactate synthase and acetolactate decarboxylase, are organized in an operon (Renna et al., J. Bacteriol (1993) 175: 3863-3875).

Furthermore, the production strain can be optimized by inactivating one or more genes the gene products whereof have an adverse effect on the 2,3-butanediol production. Examples of these are genes the gene products whereof are responsible for by-product formation. These include for example lactate dehydrogenase (lactic acid formation), acetaldehyde dehydrogenase (ethanol formation) or else also phosphotransacetylase, or acetate kinase (acetate formation).

Furthermore, the invention comprises a method for producing 2,3-butanediol by means of a production strain according to the invention.

The method is characterized in that cells of an acetoin reductase-producing production strain according to the invention are cultured in a growth medium. During this, on the one hand biomass of the production strain and on the other hand the product 2,3-BDL are formed. The formation of biomass and 2,3-BDL during this can correlate in time or else take place decoupled in time from one another. The culturing is effected in a manner familiar to those skilled in the art. For this, the culturing can be effected in shake flasks (laboratory scale) or else also by fermentation (production scale).

A method on the production scale by fermentation is preferable, wherein a fermentation volume greater than 10 l is particularly preferable and a fermentation volume greater than 300 l is especially preferable as production scale.

Growth media are familiar to those skilled in the art from the practice of microbial culturing. They typically consist of a carbon source (C source), a nitrogen source (N source) and additives such as vitamins, salts and trace elements through which the cell growth and the 2,3-BDL product formation are optimized. C sources are those which can be utilized by the production strain for forming the 2,3-BDL product. These include all forms of monosaccharides, comprising C6 sugars such as for example glucose, mannose or fructose and C5 sugars such as for example xylose, arabinose, ribose or galactose.

However, the production method according to the invention also comprises all C sources in the form of disaccharides, in particular saccharose, lactose, maltose or cellobiose.

The production method according to the invention further also comprises all C sources in the form of higher saccharides, glycosides or carbohydrates with more than two sugar units such as for example maltodextrin, starch, cellulose, hemicellulose, pectin and monomers or oligomers liberated therefrom by hydrolysis (enzymatic or chemical). Here the hydrolysis of the higher C sources can be positioned upstream of the production method according to the invention or else be effected in situ during the production method according to the invention.

Other utilizable C sources different from sugars or carbohydrates are acetic acid (or acetate salts derived therefrom), ethanol, glycerine, citric acid (and salts thereof), lactic acid (and salts thereof) or pyruvate (and salts thereof). However, gaseous C sources such as carbon dioxide or carbon monoxide are also feasible.

The C sources concerned in the production method according to the invention comprise both the isolated pure substances or else also, to increase the profitability, not further purified mixtures of the individual C sources, as can be obtained as hydrolysates by chemical or enzymatic digestion of the plant raw materials. These for example include hydrolysates of starch (monosaccharide glucose), of sugar beet (monosaccharides glucose, fructose and arabinose), of sugar cane (disaccharide saccharose), of pectin (monosaccharide galacturonic acid) or also of lignocellulose (monosaccharide glucose from cellulose, monosaccharides xylose, arabinose, mannose and galactose from hemicellulose and lignin, not a member of the carbohydrates). Furthermore, waste products from the digestion of plant raw materials, such as for example molasses (sugar beet) or bagasse (sugar cane) can also be used as C sources.

Preferable C sources for culturing the production strains are glucose, fructose, saccharose, mannose, xylose, arabinose and plant hydrolysates which can be obtained from starch, lignocellulose, sugar cane or sugar beet.

A particularly preferable C source is glucose, either in isolated form or as a component of a plant hydrolysate.

N sources are those which can be utilized by the production strain for the formation of biomass. These include ammonia, gaseous or in aqueous solution as NH₄OH or else also salts thereof such as for example ammonium sulfate, ammonium chloride, ammonium phosphate, ammonium acetate or ammonium nitrate. Also suitable as an N source are the known nitrate salts such as for example KNO₃, NaNO₃, ammonium nitrate, Ca(NO₃)₂, Mg(NO₃)₂ and other N sources such as for example urea. The N sources also include complex amino acid mixtures such as for example yeast extract, proteose peptone, malt extract, soya peptone, casamino acids, corn steep liquor (liquid or else also dried as so-called CSD) and also NZ-Amines and yeast nitrogen base.

The culturing can be effected in so-called batch mode, wherein the growth medium is inoculated with a starter culture of the production strain and then the cell growth takes place with no further feeding of nutrient sources.

The culturing can also be effected in the so-called fed batch mode, wherein after an initial phase of growth in batch mode, additional nutrient sources are fed in (feed) in order to compensate for their consumption. The feed can consist of the C source, the N source, one or more vitamins important for production, or trace elements or of a combination of the aforesaid. Here, the feed components can be metered in together as a mixture or else also separately in individual feed periods. In addition, other medium components and additives specifically increasing 2,3-BDL production can also be added to the feed. In this case, the feed can be introduced continuously or in portions (discontinuously) or else also in a combination of continuous and discontinuous feed.

The culturing is preferably effected according to the fed batch mode.

Preferable C sources in the feed are glucose, saccharose, molasses, or plant hydrolysates which can be obtained from starch, lignocellulose, sugar cane or sugar beet.

Preferable N sources in the feed are ammonia, gaseous or in aqueous solution as NH₄OH and its salts ammonium sulfate, ammonium phosphate, ammonium acetate and ammonium chloride, and furthermore, urea, KNO₃, NaNO₃ and ammonium nitrate, yeast extract, proteose peptone, malt extract, soya peptone, casamino acids, corn steep liquor and also NZ-amines and yeast nitrogen base.

Particularly preferable N sources in the feed are ammonia, or ammonium salts, urea, yeast extract, soya peptone, malt extract or corn steep liquor (liquid or in dried form).

The culturing is effected under pH and temperature conditions which favor the growth and the 2,3-BDL production of the production strain. The utilizable pH range extends from pH 5 to pH 8. A pH range from pH 5.5 to pH 7.5 is preferable. A pH range from pH 6.0 to pH 7 is particularly preferable.

The preferable temperature range for the growth of the production strain is 20° C. to 40° C. The temperature range from 25° C. to 35° C. is particularly preferable.

The growth of the production strain can be effected facultatively without oxygen input (anaerobic culturing) or else also with oxygen input (aerobic culturing). Aerobic culturing with oxygen, wherein the oxygen supply is ensured by introduction of compressed air or pure oxygen, is preferable. Aerobic culturing by introduction of compressed air is particularly preferable.

The culturing time for 2,3-BDL production is between 10 hrs and 200 hrs. A culturing time of 20 hrs to 120 hrs is preferable. A culturing time of 30 hrs to 100 hrs is particularly preferable.

Culture mixtures which are obtained by the method described above contain the 2,3-BDL product, preferably in the culture supernatant. The 2,3-BDL product contained in the culture mixtures can either be further used directly without further processing or else can be isolated from the culture mixture. For the isolation of the 2,3-BDL product, process steps known per se are available, including centrifugation, decantation, filtration, extraction, distillation or crystallization, or precipitation. These process steps can in this case be combined in any desired form in order to isolate the 2,3-BDL product in the desired purity. The degree of purity to be attained thereby is dependent on the subsequent use of the 2,3-BDL product.

Various analytical methods are available for identification, quantification and determination of the degree of purity of the 2,3-BDL product, including NMR, gas chromatography, HPLC, mass spectroscopy or also a combination of these analysis methods.

The figures show the plasmids mentioned in the examples.

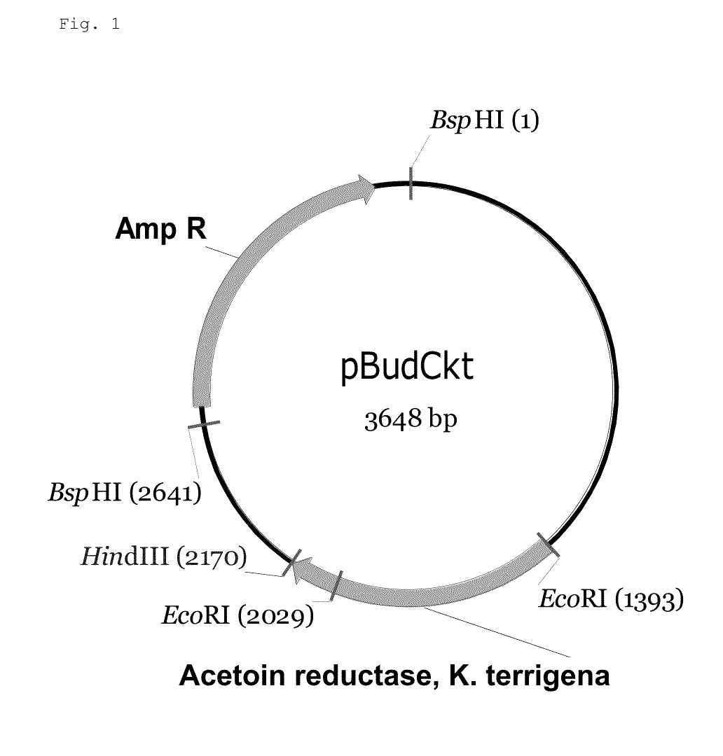

FIG. 1 shows the 3.65 kb sized acetoin reductase expression vector pBudCkt produced in example 1.

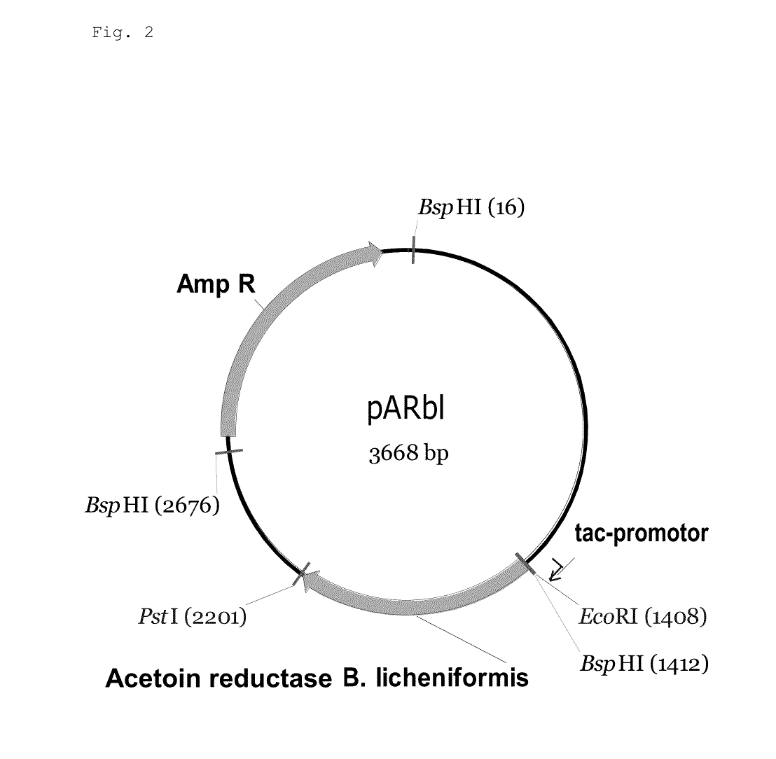

FIG. 2 shows the 3.67 kb sized acetoin reductase expression vector pARbl produced in example 1.

FIG. 3 shows the plasmid pACYC184 used in example 1.

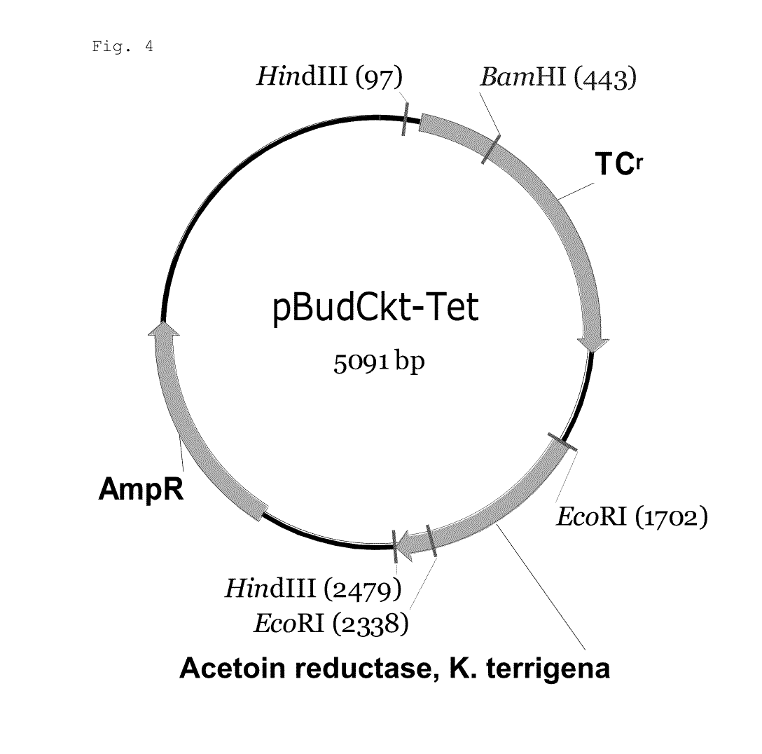

FIG. 4 shows the 5.1 kb sized expression vector pBudCkt-tet produced in example 1.

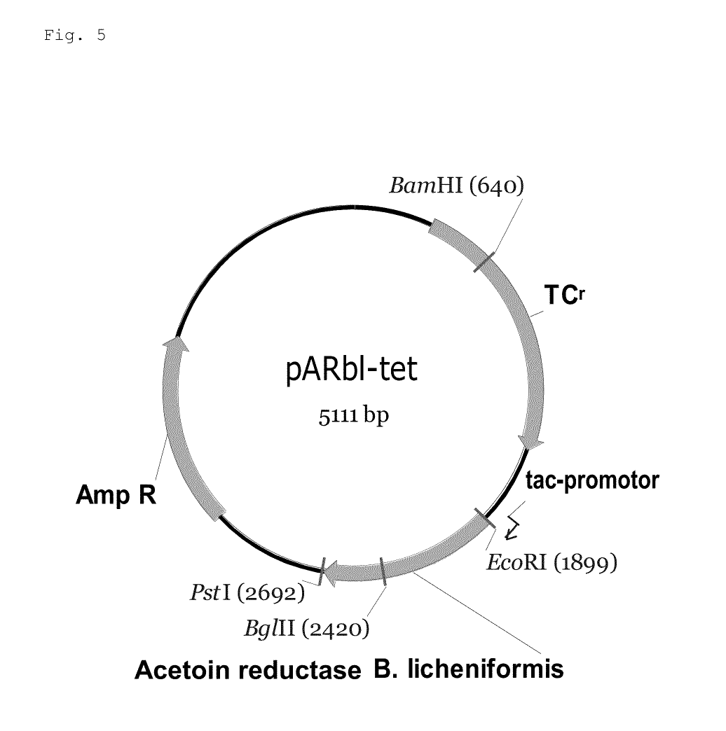

FIG. 5 shows the 5.1 kb sized expression vector pARbl-tet produced in example 1.

The invention is further explained by the following examples:

### EXAMPLE 1

Production of Acetoin Reductase Expression Vectors

The acetoin reductase genes from K. terrigena and B. licheniformis were used. The DNA sequence of the acetoin reductase gene from K. terrigena is disclosed in the “GenBank” gene database under the access number L04507, bp 2671-3440. It was isolated as a DNA fragment of 0.78 kb size in a PCR reaction (Taq DNA polymerase, Qiagen) from genomic K. terrigena DNA (strain DSM 2687, commercially available from the DSMZ Deutsche Sammlung von Mikroorganismen and Zellkulturen GmbH) with the primers BUD7f and BUD9r.

The DNA sequence of the acetoin reductase gene from B. licheniformis is disclosed in the “GenBank” gene database under the access number NC_{—}006322.1, under which the whole genome sequence of B. licheniformis is disclosed. The acetoin reductase gene can be found there in complementary form from by 2007068-2007850. It was isolated as a DNA fragment of 0.78 kb size in a PCR reaction (Taq DNA polymerase, Qiagen) from genomic B. licheniformis DNA (strain DSM 13, commercially available from DSMZ GmbH) with the primers BLar-1f and BLar-2r.

The genomic DNA used for the PCR reactions had previously been obtained in a manner known per se with a DNA isolation kit (Qiagen) from cells from the culturing of K. terrigena DSM 2687 and B. licheniformis DSM 13 in LB medium (10 g/l tryptone, 5 g/l yeast extract, 5 g/l NaCl).

Primers BUD7f (SEQ ID NO: 1) and BUD9r (SEQ ID NO:
2) had the following DNA sequences:
SEQ ID NO: 1:
5′-GAA TTC ATG CAA AAA GTC GCA CTT GTC ACC-3′
SEQ ID NO: 2:
5′-AAG CTT TTA GTT GAA TAC CAT GCC GCC GTC-3′
Primers BLar-1f (SEQ ID NO: 3) and BLar-2r (SEQ ID
NO: 4) had the following DNA sequences:
SEQ ID NO: 3:
5′-GAA TTC ATG AGT AAA GTA TCT GGA AAA ATT G-3′
SEQ ID NO: 4:
5′-CTG CAG TTA ATT AAA TAC CAT TCC GCC ATC-3′

The PCR products were then digested with Eco RI (contained in primers BUD7f and BLar-1f) and Hind III (contained in primer BUD9r) and Pst I (contained in primer BLar-2r) respectively and cloned into the expression vector pKKj. In this way, the acetoin reductase expression vectors pBudCkt (FIG. 1) and pARbl (FIG. 2) each 4.9 kb in size, were formed.

The expression vector pKKj is a derivative of the expression vector pKK223-3. The DNA sequence of pKK223-3 is disclosed in the “GenBank” gene database under the access number M77749.1. From the 4.6 kb plasmid ca. 1.7 kb were removed (bp 262-1947 of the DNA sequence disclosed in M77749.1), as a result of which the 2.9 kb expression vector pKKj was formed.

The expression vectors pBudCkt and pARbl were modified by incorporation of an expression cassette for the tetracycline resistance gene. For this, the tetracycline resistance gene was first isolated from the plasmid pACYC184 (FIG. 3). The DNA sequence of pACYC184 is accessible in the “Genbank” gene database under the access number X06403.1. By PCR (Taq DNA polymerase, Qiagen) with the primers tet1f and tet2r and subsequent digestion with Bgl II (cleavage sites contained in the primers tet1f and tet2r) the tetracycline expression cassette was isolated from pACYC184 as a 1.45 kb fragment and then cloned into the vectors pBudCkt and pARbl each cleaved with Bam HI. As a result, the expression vectors pBudCkt-tet (FIG. 4) and pARbl-tet (FIG. 5), each 6 kb in size, were formed. As shown in FIG. 4 and FIG. 5, in each case a clone was selected in which the tetracycline and the acetoin reductase expression cassettes were each oriented in the same direction.

Primers tet1f (SEQ ID NO: 5) and tet2r (SEQ ID NO:
6) had the following DNA sequences:
SEQ ID NO: 5:
5′-TCA TGA GAT CTC AGT GCA ATT TAT CTC TTC-3′
SEQ ID NO: 6:
5′-TCA TGA GAT CTG CCA AGG GTT GGT TTG CGC ATT
C-3′

### EXAMPLE 2

Expression Analysis in E. coli

Plasmid DNA from the expression vectors pBudCkt-tet and pARbl-tet was transformed by methods known per se into the E. coli strain JM105. As a control, E. coli JM105 transformed with the vector pACYC184 was used. In each case, one clone was selected and cultivated in a shake flask culture. A preculture was produced from the E. coli strains in LBtet medium (10 g/l tryptone, 5 g/l yeast extract, 5 g/l NaCl, 15 μg/ml tetracycline) (culturing at 37° C. and 120 rpm overnight). 2 ml each of preculture were used as the inoculum of a main culture of 100 ml LBtet medium (300 ml conical flask). The main cultures were shaken at 30° C. and 180 rpm until a cell density OD600 of 2.0 was reached. Then the inducer IPTG (isopropyl-β-thiogalactoside, 0.4 mM final concentration) was added and the mixture shaken overnight at 30° C. and 180 rpm.

For the analysis of the acetoin reductase expression, 50 ml of the E. coli cells were centrifuged (10 mins 15 000 rpm, Sorvall centrifuge RC5C, fitted with an SS34 rotor), the cell pellet was taken up in 2 ml KPi buffer (0.1 M potassium phosphate, 0.1 M NaCl, pH 7.0) disintegrated in a manner known per se with a so-called “French®Press” high pressure homogenizer (SLM-AMINCO) and a cell extract was isolated by centrifugation (15 mins 15 000 rpm, Sorvall SS34 rotor). Aliquots of the cell extracts were used for the spectrophotometric determination of the acetoin reductase activity. While in the control strain no activity could be measured, the specific acetoin reductase activity in cell extracts was between 70 and 122 U/mg protein, depending on the particular construct.

Spectrophotometric determination of the acetoin reductase activity: the determination of the acetoin reductase activity was performed in a manner known per se. In this case, the substrate acetoin is reduced by AR in an NADH-dependent reaction. 2,3-butanediol is formed and NADH is consumed in stoichiometric quantities. The consumption of NADH is determined photo-metrically at 340 nm. 1 U acetoin reductase activity is defined as the quantity of enzyme which reduces 1 μmol acetoin/min under test conditions.

The measurement preparation of 1 ml volume for the photometric determination of AR activity was made up of measurement buffer (0.1 M K phosphate, pH 7.0; 0.1 M NaCl and 1 mM MgCl2×7H2O), 10 μl acetoin, 0.2 mM NADH and AR-containing cell extract. The measurement temperature was 25° C. The measurement preparation was started by addition of the AR-containing cell extract and the decrease in extinction due to the consumption of NADH was measured at a wavelength of 340 nm (extinction coefficient NADH: ε=0.63×10⁴l×Mol⁻¹×cm⁻¹).

For the determination of the specific activity, the protein concentration of the cell extracts was determined in a manner known per se with the so-called “BioRad Proteinassay” from BioRad.

### EXAMPLE 3

Expression of Acetoin Reductase in Klebsiella terrigena

The original strain was Klebsiella terrigena DSM 2687. The transformation with the plasmids pBudC, pBudCkt-tet, pARbl and pARbl-tet was effected in a manner known per se analogously to the methods for transformation of E. coli familiar to those skilled in the art. As the control strain, the non-transformed wild type strain Klebsiella terrigena DSM 2687 was used.

Transformants were isolated and tested for acetoin reductase activity by shake flask culturing. For this, in each case 50 ml FM2amp medium (plasmids pBudCkt and pARbl), or FM2tet medium (plasmids pBudCkt-tet and pARbl-tet), or FM2 medium without antibiotic (K. terrigena wild type control strain) were inoculated with a transformant and incubated for 24 hrs at 30° C. and 140 rpm (Infors shaker).

FM2 medium contained glucose 60 g/l; 10 g/l; yeast extract (Oxoid) 2.5 g/l; ammonium sulfate 5 g/l; NaCl 0.5 g/l; FeSO₄×7H₂O 75 mg/l; Na₃ citrate×2H₂O 1 g/l; CaCl₂×2H₂O 14.7 mg/l; MgSO₄×7 H₂O 0.3 g/l; KH₂PO₄ 1.5 g/l; trace element mix 10 ml/l and, in the case of FM2amp, ampicillin 100 mg/l, or in the case of FM2tet, tetracycline 15 mg/l. The pH of the FM2tet medium was adjusted to 6.0 before the start of culturing.

The trace element mix had the composition H₃BO₃ 2.5 g/l; CoCl₂×6H₂O 0.7 g/l; CuSO₄×5H₂O 0.25 g/l; MnCl₂×4 H₂O 1.6 g/l; ZnSO₄×7H₂O 0.3 g/l and Na₂MoO₄×2H₂O 0.15 g/l.

The cells were analyzed as described in Example 2 for E. coli. Klebsiella cells were disintegrated with the “French®Press” and the cell extracts tested for acetoin reductase activity. The specific acetoin reductase activity in crude extracts of the various strains (determined as described in Example 2) is listed in Table 1.

TABLE 1
Comparison of the acetoin reductase activity in
recombinant K. terrigena strains

| | | Acetoin reductase | |
|---|---|---|---|
| Strain | Construct | (U/mg) | Relative activity |
| K. terrigena | — | 1.6 | 1 |
| WT | | | |
| K. terrigena | pARbl | 5.7 | 3.6 |
| pARbl | | | |
| K. terrigena | pARbl-tet | 24.9 | 15.6 |
| pARbl-tet | | | |
| K. terrigena | pBudCkt | 19.4 | 12.1 |
| pBudCkt | | | |
| K. terrigena | pBudCkt-tet | 55.7 | 34.8 |
| pBudCkt-tet | | | |

### EXAMPLE 4

2,3-BDL Production by Shake Flask Culture of Recombinant K. terrigena Strains

The culturing of the K. terrigena strains transformed with the plasmids pBudCkt-tet and pARbl-tet was effected as described in Example 3. However, the culturing time was 72 hrs. In this case, the glucose concentration was determined at intervals of 24 hrs (glucose analyzer 7100MBS from YSI) and further glucose fed in as needed from a 40% (w/v) stock solution. After a culturing time of 48 hrs and 72 hrs, samples were tested for their 2,3-BDL content. The 2,3-BDL production result is shown in Table 2.

TABLE 2
BDL production in shake flask by Klebsiella terrigena
transformants overexpressing acetoin reductase

| | K. terrigena | K. t.- | K. t.- |
|---|---|---|---|
| | WT | pBudCkt-tet | pARbl-tet |
| Time | 2,3 BDL | 2,3 BDL | 2,3 BDL |
| (hrs) | (g/1) | (g/1) | (g/1) |
| 48 | 26.4 | 31.6 | 34.6 |
| 72 | 32.0 | 41.3 | 38.6 |

As can be seen in Table 2, the overexpression of the acetoin reductase genes from K. terrigena and B. licheniformis respectively in Klebsiella terrigena resulted in an unexpectedly high increase of 20-29% in 2,3-BDL production in the shake flask culture. The increase in the 2,3-butanediol production is obviously caused by overexpression of the acetoin reductase.

The determination of the 2,3-butanediol content in culture supernatants was effected in a manner known per se by ¹H-NMR. For this, an aliquot of the culture was centrifuged (10 mins 5000 rpm, Eppendorf Labofuge) and 0.1 ml of the culture supernatant was mixed with 0.6 ml TSP (3-(trimethylsilyl)propionic acid-2,2,3,3-d₄ sodium salt) standard solution of defined content (internal standard, typically 5 g/l) in D₂O. The ¹H-NMR analysis incl. peak integration was performed according to the state of the art with an Avance 500 NMR instrument from Bruker. For the quantitative analysis, the NMR signals of the analytes were integrated in the following ranges:

TSP: 0.140-0.145 ppm (9H)

2,3-butanediol: 1.155-1.110 ppm (6H)

Ethanol: 1.205-1.157 ppm (3H)

Acetic acid: 2.000-1.965 ppm (3H)

Acetoin: 2.238-2.200 ppm (3H)

### EXAMPLE 5

2,3-BDL Production with Acetoin Reductase-Overexpressing K. terrigena Strains by Fed Batch Fermentation

“Labfors II” fermenters from Infors were used. The working volume was 1.5 l (3 l fermenter volume). The fermenters were equipped according to the state of the art with electrodes for measurement of the pO₂ and the pH and with a foam probe, which as needed regulated the metering in of an antifoam solution. The values from the measurement probes were recorded via a computer program and displayed graphically. The fermentation parameters stirrer rotation rate (rpm), aeration (supply of compressed air in vvm, volume of compressed air per volume of fermentation medium per minute), pO₂ (oxygen partial pressure, relative oxygen content calibrated to an initial value of 100%), pH and fermentation temperature were controlled and recorded via a computer program supplied by the fermenter manufacturer. Feed medium (74% glucose, w/v) was metered in via a peristaltic pump in accordance with the glucose consumption. To control foaming, a plant-based alkoxylated fatty acid ester, commercially available under the name Struktol J673 from Schill & Seilacher (20-25% v/v diluted in water), was used.

Strains used in the fermentation were the Klebsiella terrigena wild type strain (control strain from Examples 3 and 4) and the acetoin reductase-overproducing strains Klebsiella terrigena-pBudCkt-tet and Klebsiella terrigena-pARbl-tet (see Examples 3 and 4). The batch fermentation medium was FM2tet medium (see Example 3, medium without tetracycline for the K. terrigena wild type control strain).

1.35 l of the medium were inoculated with 150 ml preculture. The preculture of the strains to be fermented was produced by 24 hr shake flask culturing in batch fermentation medium. The fermentation conditions were: temperature 30° C., stirrer rotation rate 1000 rpm, aeration with 1 vvm, pH 6.0.

At regular intervals, samples were withdrawn from the fermenter for the analysis of the following parameters:

The cell density OD600 as a measure of the biomass formed was determined photometrically at 600 nm (BioRad Photometer SmartSpec™ 3000).

For the determination of the dry biomass, for each measurement point in a threefold determination 1 ml of fermentation mixture was centrifuged and the cell pellet washed with water and dried to constant weight at 80° C.

The glucose content was determined as described in Example 4.

The 2,3-BDL content was determined by NMR as described in Example 4.

After the glucose placed beforehand in the batch medium had been consumed, a 74% (w/v) glucose solution was fed in via a pump (peristaltic pump 101 U/R from Watson Marlow). In this case, the feeding rate was determined from the current glucose consumption rate.

Table 3 shows the time-dependent 2,3-BDL formation in the K. terrigena control strain and in the acetoin reductase-overproducing, recombinant Klebsiella terrigena strains.

As already observed in the shake flask experiments (Example 4), the overexpression of the acetoin reductase with the expression plasmids pBudCkt-tet and pARbl-tet leads to a significant increase in 2,3-butanediol production by 34.2% (heterologous AR gene) −43.7% (homologous AR gene), based on the maximum yield at 72 hrs fermentation time. The course of the production process is shown in Table 3.

TABLE 3
Production of 2,3-BDL in K. terrigena strains by fed batch
fermentation

| | K. terrigena | K. t.- | K. t.- |
|---|---|---|---|
| | WT | pBudCkt-tet | pARbl-tet |
| Time | 2,3 BDL | 2,3 BDL | 2,3 BDL |
| (hrs) | (g/l) | (g/l) | (g/l) |
| 24 | 60.8 | 69.7 | 71.3 |
| 27 | 68.1 | 80.8 | 76.6 |
| 31 | 69.1 | 90.2 | 86.2 |
| 48 | 81.3 | 121.2 | 110.1 |
| 51 | 80.5 | 114.0 | 113.5 |
| 55 | 86.9 | 115.8 | 118.3 |
| 72 | 91.7 | 131.8 | 123.1 |

### EXAMPLE 6

2,3-BDL Fermentation on the 330 l Scale

The strain Klebsiella terrigena-pBudCkt-tet was fermented (see Examples 4 and 5).

Production of an inoculum for the prefermenter: an inoculum of Klebsiella terrigena-pBudCkt-tet in LBtet medium (see Example 2) was produced by inoculating 2×100 ml LBtet medium, each in a 1 l conical flask, each with 0.25 ml of a glycerine culture (overnight culturing of the strain in LBtet medium, treated with glycerine in a final concentration of 20% v/v and stored at −20° C.). The culturing was effected for 7 hrs at 30° C. and 120 rpm on an Infors orbital shaker (cell density OD₆₀₀/ml of 0.5-2.5). 100 ml of the preculture were used for the inoculation of 8 l fermenter medium. Two prefermenters each with 8 l of fermenter medium were inoculated.

Prefermenter: The fermentation was performed in two Biostat® C-DCU 3 fermenters from Sartorius BBI Systems GmbH. The fermentation medium was FM2tet (see Example 3). The fermentation was effected in so-called batch mode.

2×8 1 FM2tet were each inoculated with 100 ml inoculum. The fermentation temperature was 30° C. The pH of the fermentation was 6.0 and was kept constant with the correction agents 25% NH₄OH, or 6 N H₃PO₄. The aeration was effected with compressed air at a constant flow rate of 1 vvm. The oxygen partial pressure pO2 was adjusted to 50% saturation. The regulation of the oxygen partial pressure was effected via the stirring speed (stirrer rotation rate 450-1 000 rpm). To control foaming, Struktol J673 (20-25% v/v in water) was used. After 18 hrs fermentation time (cell density OD₆₀₀/ml of 30-40), the two prefermenters were used as inoculum for the main fermenter.

Main fermenter: The fermentation was performed in a Biostat® D 500 fermenter (working volume 330 l, vessel volume 500 l) from Sartorius BBI Systems GmbH. The fermentation medium was FM2tet (Example 3). The fermentation was effected in so-called fed batch mode.

180 l FM2tet were inoculated with 16 l inoculum. The fermentation temperature was 30° C. The pH of the fermentation was 6.0 and was kept constant with the correction agents 25% NH₄OH, or 6 N H₃PO₄. The aeration was effected with compressed air at a constant flow rate of 1 vvm (see Example 4, based on the initial volume). The oxygen partial pressure pO2 was adjusted to 50% saturation. The regulation of the oxygen partial pressure was effected via the stirring speed (stirrer rotation rate 200-500 rpm). To control foaming, Struktol J673 (20-25% v/v in water) was used. In the course of the fermentation, the glucose consumption was determined by off-line glucose measurement with a glucose analyzer from YSI (see Example 4). As soon as the glucose concentration of the fermentation mixtures was ca. 20 g/l (8-10 hrs after inoculation), the metering in of a 60% w/w glucose feed solution was started. The flow rate of the feed was selected such that during the production phase a glucose concentration of 10-20 g/l could be maintained. After completion of the fermentation the volume in the fermenter was 330 l.

The analysis of the fermentation parameters was effected as described in Example 5. The content of 2,3-butanediol and the by-products acetoin, ethanol and acetate was determined by NMR (see Example 4). The lactate content was determined by HPLC in a manner known per se (see for example US 2010/0112655).

The course of the fermentation process is shown in Table 4. The yield of 128.8 g/l 2,3-butanediol achieved with the strain according to the invention Klebsiella terrigena-pBudCkt-tet was unexpectedly high and was more than 40% higher than the yield of ca. 90 g/l usually achieved with a Klebsiella terrigena wild type strain (see Example 5).

TABLE 4
Production of 2,3-BDL by fed batch fermentation
on the 330 1 scale

| | K. t.-pBudCkt-tet |
|---|---|
| Time | 2,3 BDL |
| (hrs) | (g/l) |
| 23 | 76.1 |
| 27 | 78.9 |
| 31 | 92.6 |
| 46 | 114.7 |
| 50 | 121.2 |
| 54.5 | 127 |
| 70.5 | 128.8 |

After completion of the fermentation (70.5 hrs), the fermenter broth was investigated for the content of other known fermentation products. The results are summarized in Table 5. The 2,3-butanediol content, based on the molar yield of the analytically detectable products, was 77.6% and was thus significantly higher than the yield of 49% disclosed in US 2010/0112655.

TABLE 5
Analysis of the fermentation products of the strain
K. terrigena-pBudCkt-tet

| | Content | Content | Mol content based |
|---|---|---|---|
| Product | (g/l) | (mM) | product content (%) |
| 2,3-butanediol | 128.8 | 1431 | 77.6 |
| Acetoin | 8.8 | 100 | 5.4 |
| Diacetyl | 0 | 0 | 0 |
| Acetate | 4.7 | 78.3 | 4.2 |
| Ethanol | 6.1 | 132.6 | 7.2 |
| Lactate | 9.1 | 101.1 | 5.5 |

1. A production strain for producing 2,3-butanediol producible from an original strain of the genus Klebsiella terrigena having a biosafety level of S1, wherein the production strain has an increased expression of a gene coding for the acetoin reductase enzyme compared to the original strain such that an acetoin reductase activity of the production strain is at least 3.6 to 34.8 times higher than the acetoin reductase activity of the original strain.

2. The production strain as claimed in claim 1, wherein the acetoin reductase activity of the production strain is 3.6 to 20 times higher than the acetoin reductase activity of the original strain.

3. (canceled)

4. (canceled)

5. The production strain as claimed in claim 1, wherein the gene coding for the acetoin reductase enzyme derives from a bacterium of the genus Klebsiella (Raoultella) or Bacillus.

6. The production strain as claimed in claim 1, wherein the production strain was produced from a non-genetically optimized original strain and produces an acetoin reductase in recombinant form with the result that 2,3-butanediol production (volume production, expressed in g/l 2,3-BDL) is increased compared to the non-genetically optimized original strain by at least 20%, wherein the 2,3-butanediol yield of the original strain is at least 80 g/l.

7. A method for producing 2,3-butanediol, wherein a production strain as claimed in claim 1 is cultured in a growth medium.

8. The method as claimed in claim 7, wherein a culturing is effected in a pH range from pH 5 to pH 8 and a temperature range from 20° C. to 40° C. and anaerobically or aerobically with an oxygen supply by introduction of compressed air or pure oxygen and there is a culturing time for 2,3-butanediol production of 10 hrs to 200 hrs.

9. The method as claimed in claim 7, wherein a culturing is effected in a fermentation volume greater than 300 l.

10. The production strain as claimed in claim 1, wherein the acetoin reductase activity of the production strain is 3.6 to 10 times higher than the acetoin reductase activity of the original strain.

11. The production strain as claimed in claim 2, wherein the gene coding for the acetoin reductase enzyme derives from a bacterium of the genus Klebsiella (Raoultella) or Bacillus.

12. The production strain as claimed in claim 11, wherein the production strain was produced from a non-genetically optimized original strain and produces an acetoin reductase in recombinant form with the result that 2,3-butanediol production (volume production, expressed in g/l 2,3-BDL) is increased compared to the non-genetically optimized original strain by at least 100%, wherein the 2,3-butanediol yield of the original strain is at least 80 g/l.

13. The production strain as claimed in claim 1, wherein the production strain was produced from a non-genetically optimized original strain and produces an acetoin reductase in recombinant form with the result that 2,3-butanediol production (volume production, expressed in g/l 2,3-BDL) is increased compared to the non-genetically optimized original strain by at least 100%, wherein the 2,3-butanediol yield of the original strain is at least 80 g/l.

14. The method as claimed in claim 8, wherein a culturing is effected in a fermentation volume greater than 300 l.


SEQUENCE LISTING (6 sequences)

SEQ ID NO: 1 - DNA, 30 bp, Artificial Sequence
  Synthetic DNA
gaattcatgcaaaaagtcgcacttgtcacc

SEQ ID NO: 2 - DNA, 30 bp, Artificial Sequence
  Synthetic DNA
aagcttttagttgaataccatgccgccgtc

SEQ ID NO: 3 - DNA, 31 bp, Artificial Sequence
  Synthetic DNA
gaattcatgagtaaagtatctggaaaaattg

SEQ ID NO: 4 - DNA, 30 bp, Artificial Sequence
  Synthetic DNA
ctgcagttaattaaataccattccgccatc

SEQ ID NO: 5 - DNA, 30 bp, Artificial Sequence
  Synthetic DNA
tcatgagatctcagtgcaatttatctcttc

SEQ ID NO: 6 - DNA, 34 bp, Artificial Sequence
  Synthetic DNA
tcatgagatctgccaagggttggtttgcgcattc